(19) United States
(12) Patent Application Publication
(10) Pub. No.: US 2008/0311058 A1
(43) Pub. Date: Dec. 18, 2008

(54) Stable high internal phase emulsions and compositions comprising the same

(75) Inventors: Anjing Lou, Seymour, CT (US); Alexander Lips, New Canaan, CT (US); Prem Chandar, Closter, NJ (US)

Correspondence Address: UNILEVER PATENT GROUP, 800 SYLVAN AVENUE, AG West S. Wing, ENGLEWOOD CLIFFS, NJ, 07632-3100, US

(73) Assignee: Connopco, Inc., d/b/a Unilever

(21) Appl. No.: 11/820,382

(22) Filed: Jun. 18, 2007

(51) Int. Cl. A61K 31/745; A61Q 19/00

(52) U.S. Cl. 424/59; 424/600

(57) ABSTRACT

High internal phase emulsions and end use compositions made therefrom are described. The high internal phase emulsions have superior stability and sensory characteristics and can be prepared with less than 20% by weight elastomer, an emulsifier and a steric stabilizer characterized as a polyether and/or polyglycerine cross-linked elastomer.

### FIELD OF THE INVENTION

The present invention is directed to a stable high internal phase emulsion (HIPE) and compositions comprising the same. More particularly, the invention is directed to a stable high internal phase emulsion that is suitable to serve as a base composition for a variety of end use skin care compositions. The HIPE of this invention, surprisingly, displays superior wicking capabilities when formulated with oil soluble microspheres. The same also displays excellent stability and sensory characteristics. Moreover, the HIPE of this invention may optionally be formulated with less than about 20 percent by weight elastomer.

### BACKGROUND OF THE INVENTION

Emollients including organic esters and hydrocarbons, especially petrolatum, have long been used medicinally as skin conditioning agents. These substances are second only to water as moisturizing ingredients of choice. They function primarily as an occlusive barrier. The water content of the outer layers of human skin stratum corneum is a controlling factor in the appearance of dry skin symptoms. When the stratum corneum contains an adequate amount of water within the range of ten to twenty percent, skin remains flexible. However, when the water content falls below ten percent the stratum corneum often becomes brittle and rough and can exhibit scaling and cracking.

The stratum corneum receives its water from the deep layers of the epidermis by diffusion or when it is brought into direct contact with water. The diffusion process is controlled by the water content of the skin as well as the concentration gradient. In a very dry environment, the water loss from the external skin layers can be significant and often exceed the rate of replacement by diffusion. An occlusive or semi-occlusive barrier substance placed on the surface of the skin acts to retard water loss to the environment. It also allows the skin surface to rehydrate via, for example, a diffusion mechanism.

While there are many effective and economical skin conditioning agents, they nevertheless suffer from certain disadvantages.

Often the emollient types are delivered as water-in-oil emulsions. It is difficult to attain the critical formula balance between oil and water phases to an extent sufficient to ensure long term storage stability. One part of this critical balance is the internal phase volume. A critical volume must be obtained to maximize the chemical and physical interactions, which produce and stabilize the system. If this critical volume is not balanced properly the product may suffer from viscosity change and eventual phase separation. Usually the optimum volume is quite large which limits the external phase volume size, and gives the system a draggy unfavorable slow break attribute. This critical internal phase volume restriction can reduce functionality and add unfavorable feel characteristics.

Anhydrous systems, on the otherhand, avoid the issues relating to emulsion stability. Unfortunately, other aesthetic issues arise with anhydrous systems. For example, not all oily phase materials are compatible at high concentration. Moreover, occlusive agents such as petrolatum are relatively greasy. They suffer the disadvantage of transfer onto clothing and are not easily removed from the skin by washing with soap. Neither do they allow for adequate penetration into the epidermis.

In view of the above, there is a significant need to develop compositions that are very stable and that display excellent sensory characteristics. This invention, therefore, is directed to a HIPE that is stable and that has desirable characteristics when used, for example, as a base for skin care compositions. The HIPE of this invention is stable for at least about three months (preferably at least about four months) when stored at about 45° C. Moreover, the HIPE of this invention displays excellent wicking properties when formulated with oil soluble microspheres, is easy to spread and has superior moisturizing capabilities. Particularly, the HIPE of this invention comprises a non-emulsifying elastomer, an emulsifier, and a steric stabilizer.

### ADDITIONAL INFORMATION

Efforts have been disclosed for making skin care compositions. In U.S. Pat. No. 5,833,973, skin treatment compositions with a crosslinked non-emulsifying siloxane elastomer are described.

Other efforts have been disclosed for making skin care compositions. In U.S. Pat. No. 5,908,707, cleaning articles having a high internal phase inverse emulsion are described.

Still other efforts have been disclosed for making skin care compositions. In U.S. Pat. No. 6,696,049, cosmetic compositions with emulsifying crosslinked siloxane elastomer are described.

None of the additional information above describes a HIPE that is stable for at least about three months when stored at about 45° C. whereby the HIPE comprises a non-emulsifying elastomer, an emulsifier and a steric stabilizer.

### SUMMARY OF THE INVENTION

In a first aspect, the present invention is directed to a high internal phase emulsion comprising:
(a) non-emulsifying elastomer;
(b) water;
(c) oil;
(d) emulsifier; and
(e) a steric stabilizer, the steric stabilizer being an elastomer that contributes to a final viscosity, V_{f}, of the high internal phase emulsion, wherein the high internal phase emulsion in the absence of the steric stabilizer has a stabilizer free viscosity, Vₛ, further wherein V_{f} is at least about equal to Vₛ to no more than about Vₛ+(Vₛ×0.075).

In a second aspect, the present invention is directed to a skin care composition comprising the high internal phase emulsion of the first aspect of this invention.

All other aspects of the present invention will more readily become apparent upon considering the detailed description and examples which follow.

Skin care composition, as used herein, is meant to include end use cosmetic compositions comprising the HIPE of this invention and suitable for use with humans and suitable to enhance a skin characteristic. Such a composition may generally be classified as leave-on or rinse off and is meant to include an end use composition that may be, for example, a soap; a hair care composition like a shampoo, conditioner or tonic; lipstick; a leave-on skin enhancer, or a color cosmetic. The preferred form of skin care composition of this invention is, however, a leave-on composition that in addition to displaying excellent wicking properties when formulated with oil soluble microspheres, does not display a heavy tacky and greasy feeling generally associated with conventional water-in-oil emulsions.

Skin, as used herein, is meant to include skin on the face, neck, chest, back, arms, hands, legs and scalp. Superior wicking, as used herein, is defined to mean an even distribution and absorption of sebum. Excellent sensory characteristics means yielding a comfortable and light/powdery sensation that is not heavy, tacky and greasy as well as excellent moisturizing capabilities. Steric stabilizer, as used herein means an ingredient like an elastomer that prevents coalescence of water thereby stabilizing the HIPE.

Unless explicitly stated otherwise, all ranges described herein are meant to include all ranges subsumed therein. The term comprises is meant to encompass the terms consisting essentially of and consisting of. Furthermore, unless defined otherwise, the amount of elastomer used means the amount of cross-linked polymer and carrier oil added as a mixture whereby the cross-linked polymer makes up from about 10 to about 35% by weight of the mixture.

### DETAILED DESCRIPTION OF THE PREFERRED EMBODIMENT

There is no limitation with respect to the type of non-emulsifying elastomer that may be used in this invention other than that such an elastomer is suitable for use in a composition that may be applied topically. Illustrative non-limiting examples of the types of non-emulsifying elastomers that may be used in this invention include those that have an average number (Mn) molecular weight in excess of 2,000, preferably, in excess of 5,000, and most preferably, in the range from about 10,000 to about 20 million, including all ranges subsumed therein. The term “non-emulsifying” defines a siloxane from which polyoxyalkylene units are absent. Often, the elastomers are formed from a divinyl compound which has at least two free vinyl groups, reacting with Si—H linkages of a polysiloxane backbone. Such elastomer compositions are commercially available from the General Electric Company under product designation General Electric Silicone 1229 with proposed CTFA name of Cyclomethicone and Vinyl Dimethicone Methicone Cross Polymer, delivered as 20-35% elastomer in a cyclomethicone carrier. A related elastomer composition under the CTFA name of Crosslinked Stearyl Methyl Dimethyl Siloxane Copolymer is available as Gransil SR-CYC (25-35% elastomer in a cyclomethicone carrier) from Grant Industries, Inc., Elmwood Park, N.J. The commercial products from General Electric and Grant Industries ordinarily are further processed by subjecting them to a high pressure (approximately 5,000 psi) treatment in a Sonolator with recycling in 10 to 60 passes. Sonolation achieves a resultant fluid with elastomer average particle size ranging from 0.2 to 10 micron, preferably 0.5 to 5 micron. Viscosity is preferred often when ranging between 300 and 20,000 cps at 25° C., as measured by a Brookfield LV Viscometer (size 4 bar, 60 rpm, 15 sec). In an especially preferred embodiment, a most desired non-emulsifying elastomer is a cyclomethicone/dimethicone cross-polymer made commercially available by suppliers like Dow Chemical under the name DC9045, and Shin-Etsu under the name KSG-15 elastomer gel (with about 10-25% by weight cyclomethicone/vinyl dimethicone cross-polymer in a cyclomethicone carrier).

Typically, the amount of non-emulsifying elastomer (including carrier), used in the HIPE of this invention is from about 0.05 to about 18%, and preferably, from about 2 to about 10%, and most preferably, from about 3 to about 8% by weight, based on total weight of the HIPE and including all ranges subsumed therein.

Oil suitable for use in this invention is limited only to the extent that the same can be used in a HIPE emulsion and topically applied. The oil used in the HIPE of this invention is preferably silicon-based, and particularly, one classified as a D4, D5, or D6 cyclodimethicone or a mixture thereof. Other preferred oils suitable for use in this invention include dimethicone-based oils having a viscosity from about 3 cps to about 100 cps at ambient temperature and as determined on a Ubbelohde Viscometer. Such oils may be used alone or in combination with other oils suitable for use in topical compositions.

The oil within the HIPE of this invention typically makes up from about 0.5 to about 25%, and preferably, from about 5 to about 20%, and most preferably, from about 10 to about 15% by weight of the HIPE, based on total weight of the HIPE and including all ranges subsumed therein.

In an especially preferred embodiment, less than about 60%, and preferably, less than about 50%, and most preferably, from about 2 to about 35% by weight of the total oil in the HIPE of this invention is provided as carrier with elastomer.

The emulsifier used in the HIPE of this invention is one suitable for use in topical compositions, and often, an emulsifier having an HLB of less than about 9, preferably less than about 7, and most preferably, less than about 5. Illustrative examples of the type of emulsifier that may be used in this invention include those generally classified as polyether modified silicone surfactants like PEG/PPG-20/22 butyl ether dimethicone, PEG-3 dimethicone, PEG-9 methyl ether dimethicone, PEG-10 dimethicone, mixtures thereof or the like. Such emulsifiers are made available from suppliers like Shin-Etsu and sold under the names KF-6012, KF-6015, KF-6016, and KF-6017, respectively. In an often preferred embodiment, the emulsifier used in this invention is PEG-10 dimethicone.

Typically, the emulsifier makes up from about 0.05 to about 12%, and preferably, from about 0.1 to about 10%, and most preferably, from about 0.5 to about 4% by weight of the HIPE, based on total weight of HIPE and including all ranges subsumed therein.

The steric stabilizer that may be used in this invention to prevent coalescence of water and to stabilize the HIPE of this invention is preferably an elastomer. Such a steric stabilizer is one which preferably has a refractive index of greater than about 1.4 at 25° C. Moreover, the steric stabilizer is often a cross-linked elastomer (such as a polyether and/or polyglycerine cross-linked elastomer) where the cross-linking group preferably has a chain length from about 8 to about 26 carbon atoms.

Often preferred steric stabilizers are Dimethicone/PEG-10/15 Crosspolymer in Dimethicone (KSG-210), and Dimethicone Polyglycerin-3 Crosspolymer in Dimethicone (KSG-710), mixtures thereof or the like. Such steric stabilizers are made commercially available, and especially, from suppliers like Shin-Etsu.

Typically, the amount of steric stabilizer (ie., including carrier) employed is from about 0.1 to about 25%, and preferably, from about 0.2 to about 15%, and most preferably, from about 0.5 to 8%, based on total weight of the HIPE and including all ranges subsumed therein. In a preferred embodiment, the steric stabilizer used in the HIPE of this invention is KSG-210 or a derivative or mimic thereof.

In an optional but particularly preferred embodiment, the HIPE of the present invention further comprises an alkyl modified cross-linked elastomer (such as a polyether and/or polyglycerine cross-linked elastomer) where the cross-linking group preferably has a chain length from about 8 to about 26 carbon atoms.

Illustrative examples of the types of alkyl modified cross-linked elastomers suitable for use in this invention include PEG-15/Lauryl Dimethicone Crosspolymer in Mineral Oil (KSG-310), PEG-15/Lauryl Dimethicone Crosspolymer and Isododecane (KSG-320), PEG-15/Lauryl Dimethicone Crosspolymer in Triethylhexanoin (KSG-330), PEG-10/Lauryl Dimethicone Crosspolymer and PEG 15/Lauryl Dimethicone Crosspolymer in Squalane, (KSG-340), Lauryl/Dimethicone/Polyglycerine-3 Crosspolymer in Triethylhexanoin (KSG-830), Lauryl Dimethicone/Polyglycerine-3 Crosspolymer in Squalene (KSG-840), mixtures thereof or the like.

When used, the amount of alkyl modified cross-linked elastomer (including carrier) employed is typically from about 0 to about 20%, and preferably, from about 0.1 to about 15%, and most preferably, from about 0.5 to about 5% by weight, based on total weight of HIPE and including all ranges subsumed therein.

Water will typically make up the balance of the HIPE, and should make up at least about 75% by weight of the HIPE.

Optional additives may be added to the HIPE of the present invention in order to yield a desired end use composition. For example, end use skin care compositions prepared with the HIPE of this invention may optionally contain a skin conditioning agent. These agents may be selected from humectants, exfoliants or emollients.

Humectants are polyhydric alcohols intended for moisturizing, reducing scaling and stimulating removal of built-up scale from the skin. Typical polyhydric alcohols include polyalkylene glycols and more preferably alkylene polyols and their derivatives. Illustrative are propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin and mixtures thereof. Most preferably the humectant is glycerin. Amounts of humectant may range (if used) anywhere from about 0.01 to 15%, preferably from about 0.01 to about 10%, optimally from about 0.75 to about 5% by weight.

Exfoliants suitable for use with the HIPE of the present invention may be selected from alpha-hydroxycarboxylic acids, beta-hydroxycarboxylic acids and salts of these acids.

Most preferred are glycolic, lactic and salicylic acids and their ammonium salts.

A wide variety of C₂-C₃₀ alpha-hydroxycarboxylic acids may be employed. Suitable examples include:
α-hydroxyethanoic acid
α-hydroxypropanoic acid
α-hydroxyhexanoic acid
α-hydroxyoctanoic acid
α-hydroxydecanoic acid
α-hydroxydodecanoic acid
α-hydroxytetradecanoic acid
α-hydroxyhexadecanoic acid
α-hydroxyoctadecanoic acid
α-hydroxyeicosanoic acid
α-hydroxydocosanoic acid
α-hydroxyhexacosanoic acid, and
α-hydroxyoctacosanoic acid.

When the conditioning agent is an emollient it may be selected from hydrocarbons, fatty acids, fatty alcohols and esters. Petrolatum is the most preferred hydrocarbon type of emollient conditioning agent. Other hydrocarbons that may be employed include mineral oil, polyolefins such as polydecene, and parafins such as isohexadecane (e.g. Permethyl 99® and Permethyl 101®).

Fatty acids and alcohols suitable for use often have from 10 to 30 carbon atoms. Illustrative of this category are pelargonic, lauric, myristic, palmitic, steric, isosteric, hydroxysteric, oleic, linoleic, ricinoleic, arachidic, behenic and erucic acids and alcohols.

Oily ester emollients suitable for use in end use skin care compositions made with the HIPE of this invention can be those selected from one or more of the following classes:
1. Triglyceride esters such as vegetable and animal fats and oils. Examples include castor oil, cocoa butter, safflower oil, cottonseed oil, corn oil, olive oil, cod liver oil, almond oil, avocado oil, palm oil, sesame oil, squalene, Kikui oil and soybean oil.
2. Acetoglyceride esters, such as acetylated monoglycerides.
3. Ethoxylated glycerides, such as ethoxylated glyceryl monostearate.
4. Alkyl esters of fatty acids having 10 to 20 carbon atoms. Methyl, isopropyl, and butyl esters of fatty acids are useful herein. Examples include hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate.
5. Alkenyl esters of fatty acids having 10 to 20 carbon atoms. Examples thereof include oleyl myristate, oleyl stearate, and oleyl oleate.
6. Ether-esters such as fatty acid esters of ethoxylated fatty alcohols.
7. Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol polyfatty esters, ethoxylated glyceryl monostearate, 1,2-butylene glycol monostearate, 1,2-butylene glycol distearate, polyoxyethylene polyol fatty acid ester, sorbitan fatty acid esters, and polyoxyethylene sorbitan fatty acid esters are satisfactory polyhydric alcohol esters.
8. Wax esters such as beeswax, spermaceti, myristyl myristate, stearyl stearate.

The skin care cosmetic compositions of the present invention should contain substantial levels of water. Such compositions often contain water in amounts ranging from 50 to 90%, preferably from 55 to 85% by weight.

Like the HIPEs, the end use skin care compositions of this invention are water-in-oil emulsions having a high internal (water) phase volume emulsion as described herein.

Surfactants can be a further optional additive of compositions according to the present invention. These may be selected from nonionic, anionic, cationic or amphoteric emulsifying agents. They may range in amount anywhere from about 0.1 to about 5% by weight. Illustrative nonionic surfactants are alkoxylated compounds based on C₁₀-C₂₂ fatty alcohols and acids, and sorbitan. These materials are available, for instance, from the Shell Chemical Company under the Neodol trademark. Copolymers of polyoxypropylene-polyoxyethylene, sold by the BASF Corporation under the Pluronic trademark, are sometimes also useful. Alkyl polyglycosides available from the Henkel Corporation may also be utilized for purposes of this invention.

Anionic type surfactants include fatty acid soaps, sodium lauryl sulphate, sodium lauryl ether sulphate, alkyl benzene sulphonate, mono- and di-alkyl acid phosphates and sodium fatty acyl isethionate.

Amphoteric surfactants include such materials as dialkylamine oxide and various types of betaines (such as cocoamidopropyl betaine).

Preservatives can desirably be incorporated into the compositions of this invention to protect against the growth of potentially harmful microorganisms. While it is in the aqueous phase that microorganisms tend to grow, microorganisms can also reside in the oil phase. As such, preservatives which have solubility in both water and oil are preferably employed in the present compositions. Suitable traditional preservatives are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are methyl paraben, propyl paraben, imidazolidinyl urea, sodium dehydroxyacetate and benzyl alcohol. The preservatives should be selected having regard for the use of the composition and possible incompatibilities between the preservatives and other ingredients in the emulsion. Preservatives are preferably employed in amounts ranging from 0.01% to 2% by weight of the composition.

Still other optional additives suitable for use in skin care compositions made with the HIPE of this invention include thickeners. Such thickeners are often generally classified as carboxylic acid polymers, cross-linked polyacrylate polymers, polyacrylamide polymers or the like. Typical thickeners include cross linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl-methylcellulose, hydroxypropyl cellulose, hydroxyethyl cellulose, ethyl cellulose, polyacrylamide comprising thickeners (like Sepigel™305) and hydroxymethyl cellulose. Gums suitable for the present invention include guar, xanthan, magnesium aluminum silicate (Veegum), sclerotium, carrageenan, pectin and combinations of these gums. Amounts of the thickener may range from 0.0001 to 5%, usually from 0.001 to 1%, optimally from 0.01 to 0.5% by weight, based on total weight of the skin care composition and including all ranges subsumed therein.

Sunscreens may be used (in any desirable combination) in the skin care compositions and they include those materials commonly employed to block ultraviolet light. Illustrative compounds are PABA, cinnamate and salicylate. For example, avobenzophenone (Parsol 1789®) octyl methoxycinnamate and 2-hydroxy-4-methoxyl benzophenone (also known as oxybenzone) can be used. Octyl methoxycinnamate, 2-ethylhexyl-p-methoxycinnamate, and 2-hydroxy-4-methoxy benzophenone are all commercially available. Others which may be used include octocrylene, butylmethoxydibenzoyl methane and phenylbezimidazole sulfonic acid. The exact amount of sunscreen employed in the compositions can vary depending upon the degree of protection desired from the sun's UV radiation.

Even other optional additives that may be used with the skin care compositions of this invention and they include physical scatterers (like TiO₂ and/or ZnO), skin care chelators (like EDTA), microspheres (e.g., polyethylene based spheroids sold under the name CL-2080; ethylene and methacrylate based spheroids sold under the names SPCAT-I2 and DSPCS-I2, respectively, made available by Kobo Industries), anti-inflammatory agents (including the standard steroidal and non-steroidal types), vitamins, like niacinamide, anti-oxidants, dispersants (e.g., PEG-100 stearate and/or NaCl), recorcinols and retinoids, including retinoic acid, retinal and retinyl esters.

Typically, the optional additives used to make the end use skin care composition of this invention, collectively, make up less than about 20%, and preferably, less than about 15%, and most preferably less than about 10% by weight of the skin care composition, based on total weight of the skin care composition and including all ranges subsumed therein.

Minor adjunct ingredients may also be included such as fragrances, antifoam agents, and colorants, each in their effective amounts to accomplish their respective functions.

When making the compositions of the present invention, the desired ingredients can be mixed in no particular order and usually at temperatures from about ambient to about 65° C. and under atmospheric pressure. In a preferred embodiment, however, water is added to oil.

Typically, the end use skin care compositions made with the HIPE of this invention comprise from about 50 to about 92%, and preferably, from about 60 to about 88%, and most preferably, from about 70 to about 80% by weight HIPE, based on total weight of the skin care composition and including all ranges subsumed therein.

The viscosity of the HIPE (and end use skin care composition prepared therefrom) is typically from about 5,000 to about 400,000 cps, and preferably from about 10,000 to about 300,000 cps, and most preferably from about 15,000 to about 225,000 cps taken at a shear rate of 1 S⁻¹ at ambient temperature with a strain controlled parallel plate rheometer (like those sold by T.A. Instruments under the Ares name).

In an especially preferred embodiment, the HIPE of this invention has less than about 18% by weight total elastomer. In a most especially preferred embodiment, the HIPE of this invention has from about 5 to about 13% by weight elastomer, based on total weight of the elastomer and including all ranges subsumed therein.

The packaging for the compositions of this invention can be a bottle, tube, roll-ball applicator, squeeze container or lidded jar.

The examples below are provided to illustrate the invention and are not intended to limit the scope of the claims.

### EXAMPLE 1

HIPEs were made by mixing the following components with moderate shear at ambient temperature.

HIPE

| | 1 | 2 | 3 | 4 |
|---|---|---|---|---|
| Component | Wt. % | Wt. % | Wt. % | Wt. % |
| Water | Balance | Balance | Balance | Balance |
| Glycerine | 4.5 | 4.5 | 4.5 | 4.5 |
| Emulsifier | 1-2 | 1-2 | 1-2 | 1-2 |
| Alkyl modified cross- | 1-2 | 1-2 | — | — |
| linked elastomer | | | | |
| Polyether/polyglycerine | 2-4 | 2-4 | 2-4 | 2-4 |
| cross-linked elastomer | | | | |
| Non-emulsifying | 4-8 | 4-8 | 4-8 | 4-8 |
| elastomer | | | | |
| Oil | 3-4 | 3-4 | 3-4 | 3-4 |
| Stabilizer | 0.5-1 | 0.5-1 | 0.5-1 | 0.5-1 |

The four HIPEs made according to the present invention were stored at 45° C. for four months. Visual examination of the HIPEs after storage surprisingly revealed no phase separation. A control and conventional (i.e., non-HIPE) water-in-oil emulsion suitable for topical use and made with about 26% by weight non-emulsifying elastomer was also stored under identical conditions. Visual examination of the same revealed phase separation.

### EXAMPLE 2

HIPEs similar to those described in Example 1 were made. To the HIPEs were added about 10% by weight polyethylene-based microspheres (CL-2080). Control emulsions similar to the one described in Example 1 were also made and about 5% by weight fumed silica was added to the control emulsions. Panelists applied and compared the emulsions. All panelists concluded that the HIPEs made according to this invention were easier to apply/spread than the conventional emulsions. Furthermore, all panelists concluded that the HIPEs made according to this invention were less greasy and less tacky than the controls and better at yielding superior sensory benefits, including moisturizing benefits.

Finally, sebum wicking capabilities of the HIPEs and controls described in this Example were compared (Using the test described in Liquid Transport in the Networked Microchannels of the Skin Surface by Dussaud et al., Langmuir 2003, 19, pages 7341-7345). The experiments revealed that the HIPEs made according to this example had sebum wicking capabilities that were surprisingly at least twice as good as the controls.

1. A high internal phase emulsion comprising:
(a) non-emulsifying elastomer;
(b) water;
(c) oil;
(d) emulsifier; and
(e) a steric stabilizer, the steric stabilizer being an elastomer that contributes to a final viscosity, V_{f}, of the high internal phase emulsion, wherein the high internal phase emulsion in the absence of the steric stabilizer has a stabilizer free viscosity, Vₛ, further wherein V_{f} is at least about equal to Vₛ to no more than about Vₛ+(Vₛ×0.075) and further wherein the high internal phase emulsion is a water-in-oil emulsion comprising at least about 75% by weight water.

2. The emulsion according to claim 1 wherein the non-emulsifying elastomer comprises cyclomethicone, dimethicone or both.

3. The emulsion according to claim 1 wherein the non-emulsifying elastomer is a cross-polymer comprising cyclomethicone and dimethicone.

4. The emulsion according to claim 1 wherein the non-emulsifying elastomer makes up from about 0.05 to about 18% by weight of the emulsion.

5. The emulsion according to claim 1 wherein the oil is silicon-based.

6. The emulsion according to claim 1 wherein the oil makes up from about 0.5 to about 25% by weight of the emulsion.

7. The emulsion according to claim 6 wherein less than about 60% by weight of the oil is provided as carrier for elastomer.

8. The emulsion according to claim 1 wherein the emulsifier has an HLB of less than about 9.

9. The emulsion according to claim 1 wherein the emulsifier is a polyether modified silicone.

10. The emulsion according to claim 1 wherein the emulsifier makes up from about 0.05 to about 12% by weight of the emulsion.

11. The emulsion according to claim 1 wherein the steric stabilizer has a refractive index of greater than about 1.4 at 25° C.

12. The emulsion according to claim 11 wherein the steric stabilizer is a cross-linked elastomer which is polyether and/or polyglycerine cross-linked.

13. The emulsion according to claim 12 wherein the steric stabilizer comprises dimethicone.

14. The emulsion according to claim 1 wherein the steric stabilizer makes up from about 0.1 to about 25% by weight of the emulsion.

15. (canceled)

16. The emulsion according to claim 1 wherein the emulsion comprises less than about 20% by weight elastomer.

17. The emulsion according to claim 1 wherein the emulsion comprises less than about 18% by weight elastomer.

18. The emulsion according to claim 1 wherein the emulsion comprises from about 5 to about 13% by weight elastomer.

19. The emulsion according to claim 1 wherein the emulsion is suitable for topical application to the skin.

20. A skin care composition comprising a high internal phase emulsion comprising:
(a) non-emulsifying elastomer;
(b) water;
(c) oil;
(d) emulsifier; and
(e) a steric stabilizer, the steric stabilizer being an elastomer that contributes to a final viscosity, V_{f}, of the high internal phase emulsion, wherein the high internal phase emulsion in the absence of the steric stabilizer has a stabilizer free viscosity, Vₛ, further wherein V_{f} is at least about equal to Vₛ to no more than about Vₛ+(Vₛ×0.075) and further wherein the high internal phase emulsion is a water-in-oil emulsion comprising at least about 75% by weight water.

21. The skin care composition according to claim 20 wherein the skin care composition comprises from about 50 to about 90% by weight water.

22. The skin care composition according to claim 20 wherein the skin care composition comprises from about 50 to about 92% by weight of the high internal phase emulsion.

23. The skin care composition according to claim 20 wherein the skin care composition moisturizes skin, reduces wrinkles on skin, controls oil on skin and/or lightens skin.

24. The skin care composition according to claim 20 wherein the skin care composition further comprises vitamin, antioxidant, dispersant, sunscreen, preservative, emollient, exfolliants, humectants or a mixture thereof.
